(19) United States
(12) Patent Application Publication
(10) Pub. No.: US 2015/0223747 A1
(43) Pub. Date: Aug. 13, 2015

(54) PATIENT REPORTED OUTCOME INSTRUMENT

(71) Applicant: ALVINE PHARMACEUTICALS, INC., San Carlos, CA (US)

(72) Inventor: Daniel C. Adelman, Redwood City, CA (US)

(73) Assignee: ALVINE PHARMACEUTICALS, INC., San Carlos, CA (US)

(21) Appl. No.: 14/425,932

(22) PCT Filed: Sep. 5, 2013
(86) PCT No.: PCT/US2013/058317

§ 371 Date: Mar. 4, 2015

Related U.S. Application Data
(60) Provisional application No. 61/697,360, filed on Sep. 6, 2012.

(51) Int. Cl. A61B 5/00; A61K 38/48; A61B 5/16

(52) U.S. Cl. CPC A61B 5/42; A61B 5/168; A61K 38/482; A61K 38/4873; G06Q 50/22

(57) ABSTRACT

Patient reported outcome (PRO) instruments are useful for collecting information from celiac disease patients, patients with gluten intolerance or gluten sensitivity, and patients on gluten free diets.

### FIELD OF THE INVENTION

The present invention provides patient reported outcome (“PRO”) instruments, also called questionnaires, for assessing the symptoms and severity of celiac disease, gluten intolerance, and gluten sensitivity, as well as the efficacy of treatment methods for these conditions.

### BACKGROUND OF THE INVENTION

Celiac disease is a systemic autoimmune disease involving the small intestine caused by the ingestion of gluten proteins, found, for example, in wheat, barley, and rye, which are present in a wide variety of foods. In celiac disease patients (most of whom are human leukocyte antigen (HLA) DQ2 (or DQ8)-positive), exposure of the small intestine to gluten induces an inflammatory response, leading to destruction of the villous structure of the intestine. Celiac disease can appear in early childhood, with severe symptoms including chronic diarrhea, abdominal pain and distension, and failure to thrive. In many patients, symptoms may not develop until later in life, when the disease symptoms can include fatigue, chronic diarrhea, weight loss due to malabsorption of nutrients, anemia, and neurological symptoms, as well as an enhanced risk for the development of osteoporosis and intestinal cancers.

Despite its prevalence in most population groups (about 1:100) and serious manifestations, the only option for the management of celiac disease to date is strict dietary abstinence from food containing gluten (the gluten-free diet or GFD). However, maintaining a GFD is very difficult because of the prevalence of gluten in the food supply and the difficulty of ensuring that all food consumed has been prepared in a gluten-free manner. While gluten-free food is available, it is not as readily available as other food and is usually more expensive than, and lacks the texture and consistency of, gluten-containing counterpart food. While many celiac patients try to adhere to a GFD, the percentage of them able to avoid the harmful effects of gluten ingestion is quite low.

Approximately 60% of celiac disease patients on a GFD continue to report frequent bothersome and often debilitating symptoms. Furthermore, patients on a strict GFD for 8-12 years still experience significantly more gastrointestinal symptoms than the general population as assessed by the Gastrointestinal Symptoms Rating Scale.

Alternative and GFD-complementary therapies are therefore urgently needed.

Perhaps the most promising new therapy in development involves orally administering proteases able to degrade gluten, and so referred to as “glutenases”, to a patient when the patient consumes food that may contain gluten. See PCT Patent Pub. Nos. 2003/068170; 2005/107786; and 2008/115411, each of which is incorporated herein by reference. As these therapies are developed, however, there is an increasing need for better, less intrusive methods to identify whether a patient is in remission or experiencing disease symptoms. Currently, invasive endoscopy and biopsy analysis must be used to assess whether a celiac disease patient is in remission or experiencing active disease.

Most studies exploring celiac disease using patient-reported outcomes (PROs) have focused on patients health-related quality of life (HRQL) and have used generic instruments, such as the SF-36 (Johnstone et al, 2004, Eur J Gastroenterol Hepatol. 16 (12) 1281-6; Hallert et al, 1998, Scand J Gastroenterol. (33) No. 9 933-8); EQ-5D (Gray and Papanicolas, 2010, BMC Health Services Research (10)105); the Psychological General Well-being Questionnaire (Mustalahti et al, 2002, Eff Clin Pract (5) No. 3 105-113), and the Zung Self-Rating Depression Scale (Ciacci et al, 2003, Digestive Diseases and Sciences, Vol. 48, (11) 2216-2220). Studies reporting celiac disease symptoms utilize PRO instruments developed and validated for different patient populations, such as the Gastrointestinal Symptoms Rating Scale (GSRS). The GSRS was originally developed for patients with irritable bowel and peptic ulcer disease (Svedlund et al, 1988, Dig Dis and Sciences, (33) Number 2, 129-134); it has not been validated in a celiac population and does not accurately reflect the symptom profile of celiac patients. One celiac-specific symptom measure exists: the Celiac Symptom Index (CSI; Leffler et al., 2009, Clin Gastroenterol Hepatol. (7) No. 12 1328-1334). However, this instrument was not developed in accordance with the Food and Drug Administration (FDA) guidance for patient reported outcomes (PRO) measures used to support labeling claims (FDA, 2009).

With such more effective and less intrusive methods, development of new, safe, and effective therapies could be greatly facilitated. For example, it would be very beneficial to have tools to assess whether a GFD benefited a patient and whether administration of an additional treatment method, such as glutenase therapy, provides additional symptomatic relief.

The present invention meets the need for effective celiac disease patient reported outcome instruments, which have been subject to clinical and psychometric validation, including evaluation of reliability, validity and responsiveness using appropriate statistical methods.

### SUMMARY OF THE INVENTION

In a first aspect, the present invention provides a PRO instrument in the form of a daily diary that prompts the subject to respond to one or more questions about whether, in the past 24 hours, the patient has experienced one or more episodes of diarrhea, constipation, abdominal pain, bloating, flatulence, nausea, a skin rash, fatigue, a headache, and difficulty in thinking clearly. In one embodiment, the PRO instrument also prompts the subject to provide information on one or more of any symptoms experienced, including, for example and without limitation, the number of times a symptom was experienced, the severity of the symptom, the extent to which the symptom interfered with other activities. This PRO instrument, which may be referred to herein as a “celiac disease symptom diary” or “CDSD”, has a variety of applications, including but not limited to: to measure symptoms in a clinical trial of a drug or other therapy to treat celiac disease, gluten intolerance, or gluten sensitivity, including but not limited to measuring symptoms in connection with a clinical trial endpoint; to measure symptoms to determine if a subject should be treated for celiac disease, gluten intolerance, or gluten sensitivity; to monitor the status of a patient's condition over time; to link symptoms to a biomarker, including but not limited to the damage done to the lining of the small intestine that is characteristic of celiac disease and observed by intestinal biopsy; to identify subjects that should be tested for celiac disease; to monitor the efficacy of a treatment; to determine if a subject should be treated, including but not limited to providing an economic rationale for such treatment, for celiac disease, gluten intolerance, or gluten sensitivity; and to aid a physician in determining or monitoring the health status of a patient that may have celiac disease, gluten intolerance, or gluten sensitivity.

In a second aspect, the present invention provides a PRO instrument in the form of weekly questionnaire that prompts the subject to provide a rating of the extent to which the subject's symptoms have affected the subject's daily life, social activities, emotional wellbeing, and physical functioning. In one embodiment, the PRO instrument also prompts the subject to provide the rating in the form of one of five possible rates, from “not at all” to “completely”, with optional rates of “a little”, “moderately”, and “very much” (or equivalents thereto). In various embodiments, the PRO instrument may be provided in the form of a bi-weekly or monthly questionnaire or simply to provide a point in time evaluation (no specific time period specified). This PRO instrument, called an “Impact of Celiac Disease Symptoms Questionnaire”, which may be referred to herein as an “ICDSQ”, has a variety of applications, including but not limited to: to measure symptoms in a clinical trial of a drug or other therapy to treat celiac disease, gluten intolerance, or gluten sensitivity, including but not limited to measuring symptoms in connection with a clinical trial endpoint; to measure symptoms to determine if a subject should be treated for celiac disease, gluten intolerance, or gluten sensitivity; to monitor the status of a patient's condition over time; to identify subjects that should be tested for celiac disease; to monitor the efficacy of a treatment; to determine if a subject should be treated, including but not limited to providing an economic rationale for such treatment, for celiac disease, gluten intolerance, or gluten sensitivity; and to aid a physician in determining or monitoring the health status of a patient that may have celiac disease, gluten intolerance, or gluten sensitivity.

In a third aspect, the present invention provides a PRO instrument in the form of weekly questionnaire that prompts the subject to provide a rating of the extent to which the subject's adherence to a GFD has affected the subject's quality of life in areas such as dietary choices and access to foods of interest to the subject, ability to attend social events, emotional wellbeing. In one embodiment, the PRO instrument also prompts the subject to provide the rating in the form of one of five possible rates, from “not at all” to “completely”, with optional rates of “a little”, “moderately”, and “very much” (or equivalents thereto). In various embodiments, the PRO instrument may be provided in the form of a bi-weekly or monthly questionnaire or simply to provide a point in time evaluation (no specific time period specified). This PRO instrument, called an “Impact of the Gluten-Free-Diet Questionnaire”, which may be referred to herein as an “IGFDQ”, has a variety of applications, including but not limited to determining if a subject should be treated, including but not limited to providing an economic rationale for such treatment, for celiac disease, gluten intolerance, or gluten sensitivity.

Any of the PRO instruments of the present invention may be provided in written (paper-based) or electronic (for completion on a computer, including, on a web-based) form or via telephonic interaction. In one embodiment, the PRO instruments are utilized in an interactive voice response (IVR) format. In one embodiment, the PRO instruments are utilized in a web-based format made available on social media sites. In various embodiments, the PRO instruments are utilized to screen subjects as possibly having celiac disease, gluten intolerance, or gluten sensitivity; to help diagnose patients or to identify subjects for diagnostic tests; to stratify patients for treatment; or to monitor the efficacy of a GFD or other treatment modality.

In particular, in one aspect the invention concerns a patient reported outcome questionnaire (PRO) for patients who have or are suspected of having celiac disease, gluten intolerance, or gluten sensitivity in the form of a daily diary that prompts the subject to respond to one or more questions about whether, in the past 24 hours, the patient has experienced one or more of diarrhea, constipation, abdominal pain, bloating, flatulence, nausea, a skin rash, fatigue, a headache, and difficulty in thinking clearly.

In one embodiment, the PRO further prompts the subject to provide information on one or more of any symptoms experienced, wherein such information is selected from the group consisting of the number of times a symptom was experienced, the severity of the symptom, the extent to which the symptom interfered with other activities.

In another embodiment, the PRO is essentially as shown in FIG. 1.

In another aspect, the invention concerns a patient reported outcome questionnaire (PRO) for patients who have or are suspected of having celiac disease, gluten intolerance, or gluten sensitivity in the form of weekly questionnaire that prompts the subject to provide a rating of the extent to which the subject's symptoms have affected the subject's daily life, social activities, emotional wellbeing, and physical functioning.

In one embodiment, the PRO urther prompts the subject to provide the rating in the form of one of five possible rates, from “not at all” to “completely”, with intermediate rates of “a little”, “moderately”, and “very much”.

In another embodiment, the PRO is essentially as shown in FIG. 2.

In a further aspect, the invention concerns a patient reported outcome questionnaire (PRO) for patients on a gluten free diet in the form of weekly questionnaire that prompts the subject to provide a rating of the extent to which the subject's adherence to a GFD has affected the subject's quality of life in areas such as dietary choices and access to foods of interest to the subject, ability to attend social events, emotional wellbeing.

In one embodiment, the PRO further prompts the subject to provide the rating in the form of one of five possible rates, from “not at all” to “completely”, with intermediate rates of “a little”, “moderately”, and “very much”.

In another embodiment, the PRO is essentially as shown in FIG. 3.

In yet another aspect, the invention concerns a method for assessing celiac disease symptoms in a celiac disease patient, said method comprising the steps of providing the patient with a PRO selected from the group consisting of the PROs of claim 1 or 4; collecting the questionnaires; and generating an assessment of said symptoms from said questionnaires.

In one embodiment, the celiac disease patient is a symptomatic celiac disease patient on a gluten-free diet (GFD).

In another embodiment, the patient has gastrointestinal (GI) symptoms.

In yet another embodiment, the patient additionally has non-GI symptoms.

In a further embodiment, the patient's symptoms and selected from the group consisting of abdominal pain, bloating, constipation, diarrhea, fatigue, flatulence, headache, nausea, skin rash, and problems thinking clearly.

In all embodiments, the patient may be instructed to complete the PRO once a day.

In all embodiments, the patient may be instructed to complete the PRO, recording any symptoms observed during the day, for at least two weeks, or at least three weeks, or at least four weeks, or at least five weeks, or at least six weeks, or at least eight weeks, depending on the number and nature of symptoms. Following collection of the results, the entries are analyzed for the severity and/or frequency of the symptoms reported. Based on the results (severity and/or frequency scores), a decision can be made to subject the patient to further tests and/or treatment.

In one embodiment, the method further comprises the step of administering to said patient a glutenase capable of degrading gluten.

In one embodiment, the glutenase comprises a prolyl endopeptidase (PEP).

In another embodiment, the glutenase comprises ALV001 (a modified recombinant version of the proenzyme form of cysteine endoprotease EP-B2 from barley) and ALV002 (a modified recombinant version of a prolyl endopeptidase from the bacterium Sphingomonas capsulata (SC-PEP).

In a further embodiment, the glutenase is ALV003, an orally administered, fixed dose, 1:1 ratio by weight combination of ALV001 and ALV002.

The present invention will be appreciated upon consideration of the accompanying figures and the following detailed description and examples, which are only intended to illustrate but not to limit the invention.

### BRIEF DESCRIPTION OF THE DRAWINGS

FIG. 1 provides an illustrative embodiment of a PRO of the invention termed a “Celiac Disease Symptom Diary” (CDSD). This PRO can be used to distinguish treatment and GFD effect on a symptom by symptom basis and has a 24 hour recall.

FIG. 2 provides an illustrative embodiment of a PRO of the invention termed “Impact of Celiac Disease Symptoms”. This PRO is a quality of life (QOL) instrument that reviews the past 7 days for a patient. The patient's responses will typically change throughout a treatment (as when starting a GFD or a glutenase therapy). This instrument provides the effect of symptoms on QOL parameters such as impact on daily activities, social life, well-being, and the like.

FIG. 3 provides an illustrative embodiment of a PRO of the invention termed “Impact of adherent to Gluten Free Diet”. This PRO is a QOL instrument that reviews the past 7 days and is expected to show changes over time for patients starting a GFD. This instrument also provides a static assessment of a patient's QOL while maintaining a GFD but is not expected to change for a patient already on a GFD.

FIG. 4 illustrates a Celiac Disease Symptom Diary (CDSD) Framework.

### DETAILED DESCRIPTION OF THE INVENTION

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

All publications mentioned herein are expressly incorporated herein by reference to disclose and describe the methods and/or materials in connection with which the publications are cited.

### DEFINITIONS

A “PRO instrument” as used herein, refers to a questionnaire, diary, or any other form of media that can be used to obtain verbal or recorded, e.g. written, typed or tactile, PRO data from a subject. A PRO instrument can be a health-related quality of life (HRQOL) questionnaire, such as a “celiac disease symptom diary” or “CDSD” or “CDSD PRO.” A PRO instrument can be designed to include one or more questions that have been vetted to optimize the form (e.g., graphical; textual or a combination thereof), phrasing or timing of the question to a subject in order to acquire PRO data that is more likely to be valid as compared to data acquired from a non-vetted question. A PRO instrument can be encoded as a computer-executable instruction to be performed on a PRO device.

A “clinical trial” as used herein, refers to an experimental trial or test on one or more subjects designed to determine the safety, efficacy, or basis of a label claim for a medical product. A clinical trial includes administering a medical product or a placebo to one or more subjects.

A “clinical endpoint” as used herein, refers to occurrence of, or change in, a disease, a condition, a syndrome, a symptom, a sign or a laboratory measurement in a subject that constitutes one of the target outcomes of a clinical trial.

A “surrogate endpoint” as used herein, is a measure of an effect of a medical product in a clinical trial on a human or non-human subject that correlates with a real clinical endpoint. The surrogate endpoint can be the presence, absence or change in the level of a biomarker.

A “clinical trial endpoint” as used herein, includes both a clinical endpoint and a surrogate endpoint.

A “computer-executable instruction” as used herein, refers to an instruction or a set of instructions able to operate a computer processor to achieve a desired functional result. The desired functional result can be simple, such as the storage of a value in memory, or complex, such as an invocation of an advanced programming interface (API) call that produces sophisticated functionality. The instruction set can be any suitable processor-executable instruction set, including, without limitation, a native machine architecture language, machine language, Java, JavaScript, BASIC, Visual BASIC, C, C++, C#, FORTRAN, Perl, and the like.

The term “PRO device” is used herein in the broadest sense and refers to a device which is used to administer a PRO instrument to a subject to acquire PRO data, including. A PRO device can be, without limitation, a voice recorder, a fax machine, a portable or a fixed electronic device such as a desktop computer or work station terminal.

### DETAILED DESCRIPTION

In a first aspect, the present invention provides a PRO instrument in the form of a diary, such as a daily, weekly, bi-weekly, or monthly, diary that prompts the subject to respond to one or more questions. In a particular embodiment, the PRO instrument is a daily diary that prompts the subject about whether, in the past 24 hours, the patient has experienced one or more episodes of diarrhea, constipation, abdominal pain, bloating, flatulence, nausea, a skin rash, fatigue, a headache, and/or difficulty in thinking clearly. In one embodiment, the PRO instrument also prompts the subject to provide information on one or more of any symptoms experienced, including, for example and without limitation, the number of times a symptom was experienced, the severity of the symptom, the extent to which the symptom interfered with other activities. In some embodiments, the subject is asked to identify only symptoms the subject believes associated with his or her condition (celiac disease, gluten intolerance, or gluten sensitivity), and in other embodiments, the subject is simply asked to identify the symptom (no mention of relatedness to any disease or condition).

In one embodiment, the PRO instrument, which, in a particular embodiment, may be referred to herein as a “celiac disease symptom diary” or “CDSD” or “CDSD PRO” includes one or more questions about whether the subject experienced diarrhea in a given time period, such as in the past 24 hours, and if the subject answers yes, then the CDSD may optionally include questions about one or more of the following: how many times (optionally with multiple choice answers asking about the frequency, i.e., 3 to 5 times, 6 to 10 times, and more than 10 times) the subject experienced diarrhea; the urgency of the diarrhea (optionally with multiple choice answers indicating urgency on a scale of “not urgent” to “extremely urgent” or similar phrases, with “a little urgent”, “moderately urgent”, and/or “very urgent” or the like intermediate ratings); the severity of the diarrhea (optionally with multiple choice answers indicating severity on a scale of “very mild” to “very severe” or similar phrases, with “mild”, “moderate”, and/or “severe” or the like intermediate ratings); and/or the extent to which the diarrhea interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced constipation or experienced difficulty in emptying his or her bowels in a given time period, such as in the past 24 hours, and if the subject answers yes, then the CDSD may optionally include questions about one or more of the following: how many times (optionally with multiple choice answers asking about the frequency, i.e., 3 to 5 times, 6 to 10 times, and more than 10 times, with each “time” defined as unsuccessfully attempting a bowel movement and/or having to strain to have a bowel movement, optionally with “having to strain” rated from “never” to “always” or similar terms, with intermediate ratings of “sometimes”, “often”, and/or “most of the time” or similar phrases) the subject experienced constipation; the severity of the constipation (optionally with multiple choice answers indicating severity on a scale of “very mild” to “very severe” or similar phrases, with “mild”, “moderate”, and/or “severe” or the like intermediate ratings); and/or the extent to which the constipation interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced abdominal pain in a given time period, such as in the past 24 hours, and if the subject answers yes, then the CDSD may optionally include questions about one or more of the following: how long in total pain was felt (optionally with multiple choice answers asking about the total time on a scale from “less than one hour” to “more than twelve hours” or similar times, with “one to three hours”, “four to six hours”, and/or “seven to twelve hours” or the like intermediate times); the severity of the pain (optionally with multiple choice answers indicating severity on a scale of “very mild” to “very severe” or similar phrases, with “mild”, “moderate”, and/or “severe” or the like intermediate ratings or on a scale of zero to ten, where zero is no pain and ten is the worst pain imaginable, and the subject can input any integer from zero to ten, optionally in an IVR system); and/or the extent to which the pain interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced bloating in a given time period, such as in the past 24 hours, and if the subject answers yes, then the CDSD may optionally include questions about one or more of the following: how long the bloating was felt (optionally with multiple choice answers asking about the total time on a scale from “less than one hour” to “more than twelve hours” or similar times, with “one to three hours”, “four to six hours”, and/or “seven to twelve hours” or the like intermediate times); the severity of the bloating (optionally with multiple choice answers indicating severity on a scale of “very mild” to “very severe” or similar phrases, with “mild”, “moderate”, and/or “severe” or the like intermediate ratings); and/or the extent to which the bloating interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced flatulence in a given time period, such as in the past 24 hours, and if the subject answers yes, then the CDSD may optionally include questions about the extent to which the flatulence interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced nausea in a given time period, such as in the past 24 hours due to his or her condition, and if the subject answers yes, then the CDSD may optionally include questions about one or more of the following: how long the nausea was felt (optionally with multiple choice answers asking about the total time on a scale from “less than one hour” to “more than twelve hours” or similar times, with “one to three hours”, “four to six hours”, and/or “seven to twelve hours” or the like intermediate times); the severity of the nausea (optionally with multiple choice answers indicating severity on a scale of “very mild” to “very severe” or similar phrases, with “mild”, “moderate”, and/or “severe” or the like intermediate ratings); and/or the extent to which the nausea interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced a skin rash in a given time period, such as in the past 24 hours, and if the subject answers yes, then the CDSD may optionally include questions about how the subject would rate the severity of the rash (optionally with multiple choice answers indicating severity on a scale of “very mild” to “very severe” or similar phrases, with “mild”, “moderate”, and/or “severe” or the like intermediate ratings); and/or about the extent to which the skin rash interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced fatigue, which may alternately be termed “tiredness”, in a given time period, such as in the past 24 hours, and if the subject answers yes, then the CDSD may optionally include questions about one or more of the following: how long the fatigue was felt (optionally with multiple choice answers asking about the total time on a scale from “less than one hour” to “more than twelve hours” or similar times, with “one to three hours”, “four to six hours”, and/or “seven to twelve hours” or the like intermediate times or more simply as “a little of the time” to “all of the time” or like phrases, with “some of the time” and “most of the time” or the like intermediate ratings); the severity of the fatigue (optionally with multiple choice answers indicating severity on a scale of “very mild” to “very severe” or similar phrases, with “mild”, “moderate”, and/or “severe” or the like intermediate ratings); and/or the extent to which the fatigue interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced a headache in a given time period, such as in the past 24 hours, and if the subject answers yes, then the CDSD may optionally include questions about one or more of the following: how long in total the subject experienced headache (optionally with multiple choice answers asking about the total time on a scale from “less than one hour” to “more than twelve hours” or similar times, with “one to three hours”, “four to six hours”, and/or “seven to twelve hours” or the like intermediate times); the severity of the headache (optionally with multiple choice answers indicating severity on a scale of “very mild” to “very severe” or similar phrases, with “mild”, “moderate”, and/or “severe” or the like intermediate ratings or on a scale of zero to ten, where zero is no pain and ten is the worst pain imaginable, and the subject can input any integer from zero to ten, optionally in an IVR system); and/or the extent to which the headache interfered with other activities (optionally with multiple choice answers indicating severity on a scale of “not at all” to “completely” or similar phrases, with “a little bit”, “moderately”, and/or “very much” or the like intermediate ratings).

In one embodiment, the CDSD includes one or more questions about whether the subject experienced difficulty thinking clearly in a given time period, such as in the past 24 hours, and if the subject answers yes, then the CDSD may optionally include questions about one or more of the following: the severity of the difficulty in thinking clearly (optionally with multiple choice answers indicating severity on a scale of “very mild” to “very severe” or similar phrases, with “mild”, “moderate”, and/or “severe” or the like intermediate ratings); and/or the extent to which the difficulty in thinking clearly interfered with other activities (optionally with multiple choice answers indicating severity on a scale of “not at all” to “completely” or similar phrases, with “a little bit”, “moderately”, and/or “very much” or the like intermediate ratings).

It is important to understand that the questions and inquiries listed herein, or included in the illustrative CDSDs, can be listed in any order, can be combined with questions from other CDSDs and/or additional questions not expressly listed herein.

The CDSD has a variety of applications, including but not limited to: to measure symptoms in a clinical trial of a drug or other therapy to treat celiac disease, gluten intolerance, or gluten sensitivity, including but not limited to measuring symptoms in connection with a clinical trial endpoint; to measure symptoms to determine if a subject should be treated for celiac disease, gluten intolerance, or gluten sensitivity; to monitor the status of a patient's condition over time; to link symptoms to a biomarker; to identify subjects that should be tested for celiac disease; to monitor the efficacy of a treatment; to determine if a subject should be treated, including but not limited to providing an economic rationale for such treatment, for celiac disease, gluten intolerance, or gluten sensitivity; and to aid a physician in determining or monitoring the health status of a patient that may have celiac disease, gluten intolerance, or gluten sensitivity.

An embodiment of the CDSD was developed in line with the US FDA Guidance (2009), in patients with symptomatic celiac disease while on a GFD. As noted above, about two-thirds of such patients have been reported to have celiac-associated symptoms on a GFD. The CDSD provides an assessment of the frequency and severity of such symptoms.

Adult patients with biopsy-documented celiac disease who had experienced GI or non-GI celiac disease-related symptoms within the prior three months despite being on a GFD were eligible for enrollment; patients with refractory celiac disease, IBS, IBD, eosinophilic esophagitis, microscopic colitis, or other serious medical conditions were excluded. The CDSD was designed to collect data on symptoms common to celiac disease: abdominal pain, bloating, constipation, diarrhea, fatigue, flatulence, headache, nausea, skin rash, and problems thinking clearly. The presence or absence of a symptom attributed to celiac disease in the past 24 hours was first established. If a patient reported that a symptom had been experienced over the past 24 hours, a series of follow-on questions were then asked to establish the severity and impact on daily functioning related to that symptom. Only patients who completed the CDSD on at least four of the seven study days were considered eligible for the symptom diary analysis.

202 patients were enrolled in the study. 87% were between 18-60 years old. 75% had been diagnosed for over 1 year, and 25% for between six and twelve months. 80% were women; 98% were Caucasian. 85% reported being GFD adherent all the time, 14% most of the time. The mean number of days of data collection was 6.5. Greater than 90% reported at least one day of symptoms, and 44% reported 5-10 symptoms in the 7-day period. For each symptom, the percent of patients reporting that symptom at least once during the reporting period was: flatulence 63%, abdominal pain 62%, fatigue 56%, bloating 54%, diarrhea 46%, constipation 42%, nausea 35%, difficulty thinking 35%, headache 31%, and skin rash 17%. 76% of patients self-reported worst symptoms that were moderate to very severe (35% moderate, 41% severe or very severe) and affected perceived quality of life (i.e., greater than 20% reported moderate-to-complete daily disruption of social, work, and physical activities, and emotional wellbeing).

This study demonstrated the utility of the CDSD PRO in symptomatic celiac disease patients on a GFD and showed that both gastrointestinal (GI) and non-GI symptoms have a major effect on patient's quality of life.

Another embodiment of the CDSD PRO of the invention is provided in the form of an IVR system. In one embodiment, the patient is instructed to call a number, and the number called responds with a greeting and instructions. An illustrative greeting might include one or more of the following: asking the caller to listen to each question carefully before responding; advising that, if there is no response to a question, the question will be repeated after a short period of time; asking the caller to press the appropriate number key on the telephone key pad to indicate the response; advising the caller that he or she should respond to all of the questions asked and not hang up the telephone until being advised, for example, by a phrase such as “Thank you and goodbye”; and advising that, if the call is interrupted or incomplete in any manner, then it may have to be repeated.

In some embodiments, the caller is advised how to put the system on hold, i.e., by pressing the star key and awaiting further instructions. In one embodiment, the caller is advised that the system will stay on hold for up to five minutes or some pre-selected longer period of time and may be restarted by pressing a key, such as the star key.

In some embodiments, the greeting includes a welcome and instructions that the diary should be completed daily and is focused on the previous twenty-four hours.

In one IVR system, the caller is asked about diarrhea with a yes/no (press 1/press 2) question and if the caller responds “yes”, the system inquires about the frequency, i.e., once/twice/three times or more (press 1/press 2/press 3).

In one IVR system, the caller is asked whether he or she has had a complete spontaneous bowel movement (defined as a sensation of fully emptying the bowels with no strain) with a yes/no (press 1/press 2) question and if the caller responds “yes”, the system inquires about the number, i.e., once/twice/three times or more (press 1/press 2/press 3).

In one IVR system, the caller is asked whether he or she has had abdominal pain with a yes/no (press 1/press 2) question and if the caller responds “yes”, the system inquires about the severity by asking for a rating between 0 and 10, on a scale of zero to ten, where zero is no pain and ten is the worst pain imaginable, and the subject can input any number from zero to ten (press 0 to press 10).

In one IVR system, the caller is asked whether he or she has had bloating with a yes/no (press 1/press 2) question and if the caller responds “yes”, the system inquires about the severity by asking for a rating between mild and very severe or similar terms, on a scale of one to five, where one (press 1) is very mild and five (press 5) is very severe, and the subject can press 2, 3, or 4 to provide ratings of “mild”, “moderate”, and/or “severe” or similar terms.

In one IVR system, the caller is asked whether he or she has had nausea with a yes/no (press 1/press 2) question and if the caller responds “yes”, the system inquires about the severity by asking for a rating between mild and very severe or similar terms, on a scale of one to five, where one (press 1) is very mild and five (press 5) is very severe, and the subject can press 2, 3, or 4 to provide ratings of “mild”, “moderate”, and/or “severe” or similar terms.

In one IVR system, the caller is asked whether he or she has had tiredness with a yes/no (press 1/press 2) question and if the caller responds “yes”, the system inquires about the severity by asking for a rating between mild and very severe or similar terms, on a scale of one to five, where one (press 1) is very mild and five (press 5) is very severe, and the subject can press 2, 3, or 4 to provide ratings of “mild”, “moderate”, and/or “severe” or similar terms.

IVR systems of the invention can have all six of the foregoing questions or any sub-combination thereof. In another embodiment, the six foregoing questions, or a subset thereof, if provided in written or electronic form (not via an IVR system). In some embodiments, a composite score of all questions in the survey is generated and transmitted to a physician or other health care service provider. Typically, the composite score will be compared to a baseline score, and a decision made based on whether a patient's score is higher or lower than the baseline score. The baseline score can be selected by any of a variety of means, including but not limited to population studies.

PRO instruments, including those implemented with IVR systems, have a wide variety of useful applications. In one embodiment, the PRO instrument is used to define an endpoint in a clinical trial for a drug to treat celiac disease, gluten intolerance, or gluten sensitivity that supports a label with language that indicates a higher proportion of patients receiving the drug had a reduction in the frequency of symptoms and/or symptom severity, or both, as measured by the instrument, than patients receiving placebo or any other reference treatment.

This CDSD PRO instrument of the invention can also be used to correlate symptoms and symptom severity with other biological markers. For example, duodenal mucosal morphometry, measured in intestinal samples removed from a patient by endoscopy, with one measure being the villus height crypt depth ratio (Vh:Cd), is a key diagnostic feature of celiac disease, and a favorable change in the Vh:Cd ratio can be a primary endpoint in a clinical trial of a drug for the treatment of celiac disease. Another indicator of celiac disease is the presence of intraepithelial lymphocytes (IEL), and a favorable change in IEL is an indicator that a drug for the treatment of celiac disease is having a therapeutic effect. Once the CDSD PRO instrument has been demonstrated to correlate with an established biomarker, it can then be used in place of that biomarker. This can be a significant advantage, especially when the biomarker measurement is expensive or difficult for the patient, such as the endoscopic procedure used to measure Vh:Cd.

The CDSD PRO instrument can also be used by physicians to assess whether a therapy is needed, expected to provide or providing benefit to a patient. In one embodiment, a patient is given a diary (paper or electronic) on which they record the presence of each of a set of symptoms (i.e., four, six, ten, or more symptoms) daily for a period of at least one to two weeks or more (i.e., a month). In one embodiment, no symptom is scored “0”, and if a symptom occurs, the patient records “1” for mild, “2” for moderate, “3” for severe, “4” for very severe (or similar scoring). Optionally, one could also have the patient score the impact of symptoms on activities of daily living (ADLs) and emotional wellbeing as well, with no impact scored as a “0”, small impact scored as a “1”, moderate impact scored as a “2”, large impact scored as a “3”, and complete impact scored as a “4”. The patient returns the completed chart to physician for scoring. The physician can use any of a variety of scoring methods; for example, the physician could score based on one or more of the following: the number of days with at least one symptom, the number of days with at least one moderate or severe symptom, the number of days when symptoms impact ADLs or emotional well-being, the total sum of symptom scores, and the total sum of impact scores. The score for a patient would be compared to a baseline score, and the patient may be administered a therapy if the score so indicates. Once the patient is on a therapy, a series of diaries are prepared by the patient over time and scored by the physician to determine if the therapy is positively impacting the patient's symptoms.

In a second aspect, the present invention provides a PRO instrument in the form of a questionnaire, such as a weekly questionnaire, which may be called an “ICDSQ” or “ICDSQ PRO”, that prompts the subject to provide a rating of the extent to which the subject's symptoms have affected the subject's daily life, social activities, emotional wellbeing, and physical functioning. In one embodiment, the PRO instrument also prompts the subject to provide the rating in the form of one of five possible rates, from “not at all” to “completely” or similar terms, with optional intermediate rates of “a little”, “moderately”, and/or “very much” (or equivalents thereto). In various embodiments, the PRO instrument may be provided in the form of a bi-weekly or monthly questionnaire or simply to provide a point in time evaluation (no specific time period specified). This ICDSQ has a variety of applications, including but not limited to: to measure the impact of symptoms on a patient's daily life in a clinical trial of a drug or other therapy to treat celiac disease, gluten intolerance, or gluten sensitivity, including but not limited to measuring the impact of symptoms on a patient's daily life in connection with a clinical trial endpoint; to measure the impact of symptoms on a patient's daily life to determine if a subject should be treated for celiac disease, gluten intolerance, or gluten sensitivity; to monitor the status of a patient's condition over time; to identify subjects that should be tested for celiac disease; to monitor the efficacy of a treatment; to determine if a subject should be treated, including but not limited to providing an economic rationale for such treatment, for celiac disease, gluten intolerance, or gluten sensitivity; and to aid a physician in determining or monitoring the health status of a patient that may have celiac disease, gluten intolerance, or gluten sensitivity.

In one embodiment, the ICDSQ inquires about one or more of the following: the extent to which daily life has been disrupted; sleep has been disrupted; symptoms have interfered with work or school and/or rendered one less effective at work or school; symptoms have negatively affected social activities or enjoyment of them and/or daily interactions with family and/or friends; symptoms have caused embarrassment, anxiety, anger, annoyance, and/or sadness or depression; and/or symptoms have interfered with one's ability to participate in physical activity or the enjoyment of such activity.

In a third aspect, the present invention provides a PRO instrument in the form of a questionnaire, such as a weekly questionnaire, which may be called an “IGFDQ” or “IGFDQ PRO” that prompts the subject to provide a rating of the extent to which the subject's adherence to a GFD has affected the subject's quality of life in areas such as dietary choices and access to foods of interest to the subject, ability to attend social events, emotional wellbeing. In one embodiment, the PRO instrument also prompts the subject to provide the rating in the form of one of five possible rates, from “not at all” to “completely”, with optional rates of “a little”, “moderately”, and “very much” (or equivalents thereto). In various embodiments, the PRO instrument may be provided in the form of a bi-weekly or monthly questionnaire or simply to provide a point in time evaluation (no specific time period specified). The IGFDQ has a variety of applications, including but not limited to determining if a subject should be treated, including but not limited to providing an economic rationale for such treatment, for celiac disease, gluten intolerance, or gluten sensitivity.

In one embodiment, the IGFDQ inquires about one or more of the following: whether the GFD limited the variety of foods the caller enjoyed; whether the caller found it difficult to find foods because of the GFD; whether the caller was limited in the number of places the caller could eat due to the GFD; whether the caller avoided social events and/or eating out in restaurants due to the GFD; whether the caller experienced one or more of embarrassment, anxiety, anger, envy, annoyance, sadness, restriction on freedom, a feeling of being left out, feeling like a burden, loss, and/or being down or depressed due to the GFD; whether the caller's daily interactions with family and/or friends were negatively impacted by the GFD; and/or whether the GFD interfered with the caller's enjoyment of social events.

Any of the PRO instruments of the present invention may be provided in written (paper-based) or electronic form, including, without limitation, recordation on a computer readable medium, completion on a computer, including, in a web-based form or via telephonic interaction. In one embodiment, the PRO instruments are utilized in an interactive voice response (IVR) format. In one embodiment, the PRO instruments are utilized in a web-based or cloud-based format made available on a social media cite or otherwise. The answers can be recorded in similar formats, including in the form of paper-based or computer-readable reports. In various embodiments, the PRO instruments are utilized to screen subjects as possibly having celiac disease, gluten intolerance, or gluten sensitivity; to diagnose patients or to identify subjects for diagnostic tests; to stratify patients for treatment; or to monitor the efficacy of a GFD or other treatment modality.

In all aspects, the information recorded in the PRO instrument can be analyzed. The results of such analysis are useful in more clearly understanding the condition and status of a particular patient, and enable a practitioner to make informed decisions about further testing and/or treatment options. The PRO instruments are analyzed based on the total number and severity of symptoms recorded during the recordation and/or observation period. Thus frequency and/or severity scores can be calculated for every day, if the patient is requested to complete the PRO instrument daily, and for the observation period, e.g. two weeks, if the PRO instrument is completed for a period of two weeks. When a physician receives the report, he/she will be able to better categorize and manage the patient. If the decision is to subject the patient to treatment and/or lifestyle change, such as dietary restriction, the patient may be requested to complete a PRO instrument for an additional period of time in order to assess the efficacy of the treatment or lifestyle change. Depending on the results of the follow up monitoring, the treatment may be continued, adjusted, discontinued, combined with additional treatments and/or dietary restrictions, etc.

Further details of the invention are illustrated by the following non-limiting Examples.

### Example 1

### Celiac Disease Symptom Diary (CDSD)

The CDSD is a daily diary administered via an Interactive Voice Response System (IVRS) across a seven-day period. The CDSD assesses 10 common celiac symptoms: diarrhea, constipation, abdominal pain, bloating, gas, nausea, skin rash, fatigue, headache and difficulty thinking clearly. The presence or absence of each of the 10 symptoms over the previous 24 hours is first reported. If the respondent indicates the presence of a particular symptom, contingent follow-up questions assess symptom severity and impact on daily functioning. The number of follow-up questions varies from one to four depending to the symptom assessed. Response options vary from a 0-10 numeric rating scale to 0-4, 1-4 or 1-5 adjectival likert scales.

Patients are asked to complete the CDSD for 7 consecutive days around the same time each evening before going to bed. The diary can only be completed one time in any 24 hour period. If patients miss a day completing the diary, the patient can complete it the following day, but cannot provide information for the day that they missed.

Scoring of the CDSD includes a weekly severity score for each symptom and a total weekly symptom severity score. To calculate the weekly severity domain score for each symptom, each associated severity item is first transformed to exhibit a minimum score of 0 and a maximum score of 10.

The transformation is conducted as follows. For items with a minimum rating scale score of 0, one divides by the maximum assignable rating and then multiplies by 10. For items with a minimum rating scale score of 1, subtract one, divide by the maximum possible rating score minus one, and multiply by 10; i.e. 10*(score-to 1)/(max scale rating −1). The minimum and maximum values refer to the lowest and highest values on the rating scale, and not on participant data.

The weekly severity domain score for each symptom is calculated as the daily mean of the associated severity items summed across the 7 days. Thus the weekly severity domain scores for each symptom range from 0-70.

To calculate the total weekly symptom severity score, all weekly symptom severity domain scores are summed, and the total is divided by 10. If 9 symptoms are included in the endpoint, the total weekly symptom severity score ranges from 0-63.

Regarding missing data, where participants have dialed in and completed the CDSD but there are items unanswered (as may occur due to the absence of the symptom), those item scores can be simply recorded as missing. In some applications, no imputation methods are applied if the diary is not completed on any particular day. In some embodiments, if the diary is completed on <4 of 7 consecutive days, it cannot be scored, and that week's data would be considered missing.

### Example 2

### Administration of a CDSD

A physician administers the CDSD to a celiac disease patient under her care.

The patient is seen for an annual check-up. In order to determine the current disease status, the physician measures tTG-IgA celiac disease serologies, as well as the CDSD to assess symptoms on a GFD.

The patient receives an invitation to access the CDSD via a secure cloud-based site. The patient logs on once per day, and addresses each serious of questions asked. The patient continues to fill out the daily questionnaires until he/she is notified that sufficient data has been captured, and that the information will be analyzed and sent to his/her physician. The required duration of responses is based on frequency and severity of symptoms recorded. The minimum time period to collect symptom responses is one week, but may extend to 2 weeks, 4 weeks, 6 weeks, or 8 weeks, or longer based on frequency and consistency of symptoms recorded.

The physician receives a score card report of the patient's symptoms. In this illustrative example, the patient experienced the following symptoms, with frequency, severity, interference with activity, and other identifying information listed. Resulting item score is listed.
Day 1
   Diarrhea
      Frequency: 5× during the day (score=2)
      Urgency: Extremely urgent (score=4)
      Severity: Severe (score=4)
      Interference with activities: Very much (score=3)
   Bloating
      Duration: 4.5 hours (score=3)
      Severity: Moderate (score=3)
      Interference with activities: Moderate (score=2)
Day 5
   Bloating
      Duration: 45 minutes (score=1)
      Severity: Very mild (score=1)
      Interference with activities: A little bit (score=1)
   Abdominal Pain
      Duration: 2 hours of pain (score=2)
      Severity of pain—Moderate (score=3)
      Interference with activities: Moderate (score=2)
   Headache
      Duration: 2 hours (score=2)
      Severity: Mild (score=2)
      Interference with activities: A little bit (score=1)
Day 6
   Diarrhea
      Frequency: 8× during the day (score=3)
      Urgency: Moderately urgent (score=3)
      Severity: Severe (score=4)
      Interference with activities: Completely (score=4)
Day 9
   Tiredness
      Duration: 8 hours (score=4)
      Severity: Moderate (score=3)
      Interference with activities: Moderate (score=2)
Day 13
   Nausea
      Duration: One hour (score=2)
      Severity: Mild (score=2)
      Interference with activities: A little bit (score=1)

For clarity, the following symptoms were not reported: Constipation, Passing Gas, Difficulty thinking clearly. Also, no symptoms were reported on Days 2,3,4,7,8,10,11,12, and 14

An example of one embodiment of a score report is listed below:

Reporting Period: 14 days

Symptoms Dashboard Report to Physician:

| | | Duration or | | | Interference | Total Sum |
|---|---|---|---|---|---|---|
| | | Number of | | | with | of Scores |
| | Number | Episodes (if | Highest | Severity; | Activities; | by |
| | of | applicable); Sum: | Level of | Sum | Sum | Symptoms |
| | Symptom | duration/episode | Urgency (if | severity | interference | (Across |
| Symptom | Days | score | applicable) | score | score | Domains) |
| Diarrhea | 2 | 13 episodes; | Extremely | Severe; | Completely; | Sum = 27 |
| | | sum = 5 | urgent; | sum = 8 | sum = 7 | |
| | | | sum = 7 | | | |
| Bloating | 2 | >5 hours; sum = 4 | N/A | Moderate; | Moderate; | Sum = 11 |
| | | | | sum = 4 | sum = 3 | |
| Abdominal | 1 | 2 hours; sum = 2 | N/A | Moderate; | Moderate; | Sum = 7 |
| Pain | | | | sum = 3 | sum = 2 | |
| Headache | 1 | 2 hours; sum = 2 | N/A | Mild; | A little bit; | Sum = 5 |
| | | | | sum = 2 | Sum = 1 | |
| Tiredness | 1 | 8 hours; sum = 4 | N/A | Moderate; | Moderate; | Sum = 9 |
| | | | | sum = 3 | sum = 2 | |
| Nausea | 1 | 1 hour; sum = 2 | N/A | Mild; | A little bit; | Sum = 5 |
| | | | | sum = 2 | sum = 1 | |
| Total | 8 | Duration/Episodes | Urgency | 2 Severe; | 1 Complete; | Total |
| | Symptom | Category Sum = 19 | Category | 3 moderate; | 1 Very | Sum = 64 |
| | Days | | Sum = 7 | 2 mild; 1 | much, 3 | |
| | | | | very mild. | moderate; 3 | |
| | | | | Severity | A little bit. | |
| | | | | Category | Interference | |
| | | | | Sum = 22 | Category | |
| | | | | | Sum = 16 | |

Note, it is also possible to calculate Averages. Averages can be calculated by taking the sum total by symptom or category and dividing by number of days in the reporting period. For example, the sum of points assigned to diarrhea was 27. The total number of days=14, therefore, the daily average for diarrhea=27/14=1.93 or ˜2. In another example, the sum of points assigned to the Severity category was 22. The total number of symptom days reported were 8, therefore, the average symptom severity=22/8=2.75. One could also take the Severity category number of 22 and divide by number of days (14)=22/14=1.57 OR take the Severity category number of 22 and divide by the total possible symptom days 14 days×10 symptoms=140 symptom days. Therefore the percentage of symptom days=22/140=0.16

When the physician receives the report, he/she will be able to better categorize and manage the patient. In this example, a patient on an attempted GFD has over a 2 week period, experienced 8 symptom days (2 severe, 3 moderate, 2 mild, and 1 very mild). Symptoms include diarrhea, bloating, abdominal pain, headache, tiredness, and nausea. These symptoms' impact on the patient's activities were: complete (1), very much (1), moderate (3), and a little bit (3). This information would enable the physician to have a more clear understanding of the patient including:
Ongoing symptoms on GFD means patient is still being exposed to some gluten
Presence of moderate and severe symptoms likely indicate that patient has intestinal inflammation and ongoing celiac disease autoimmune reaction
Patient may be a candidate for a follow up biopsy
Patient would likely be a candidate for a celiac disease therapeutic agent to be combined with an attempted GFD
Patient likely has higher risk of long-term consequences of celiac disease (anemia, osteoporosis/osteopenia, liver disease, kidney disease, neurologic disease, lymphoma, etc.)

After additional intervention, patient should be followed at set intervals to monitor disease and determine response to therapy. Patient should follow-up with physician and physician should consider having patient take CDSD again in future (e.g. 6 months or 1 year). New score can be compared with existing score. Deterioration of score means patient may be worsening (disease control worse). Improved score may mean patient's celiac disease is improving on GFD. For example patient's baseline score=64 with 8 symptom days in this example, and patient is placed on a glutenase. Patient has follow-up CDSD at time 6 months and score is now 20 and symptom days are now 4. This is valuable evidence that the patient is improving on a glutenase. Physician would continue glutenase. However, if the follow-up score on a glutenase is worse e.g. 108 with 12 symptom days, this may mean that the patient may have some other process evolving (RCD, SBBO, etc.). This deterioration would likely warrant further evaluation and additional biopsy and testing.

1. A patient reported outcome questionnaire (PRO) for patients who have or are suspected of having celiac disease, gluten intolerance, or gluten sensitivity in the form of a daily diary that prompts the subject to respond to one or more questions about whether, in the past 24 hours, the patient has experienced one or more of diarrhea, constipation, abdominal pain, bloating, flatulence, nausea, a skin rash, fatigue, a headache, and difficulty in thinking clearly.

2. The PRO of claim 1 that further prompts the subject to provide information on one or more of any symptoms experienced, wherein such information is selected from the group consisting of the number of times a symptom was experienced, the severity of the symptom, the extent to which the symptom interfered with other activities.

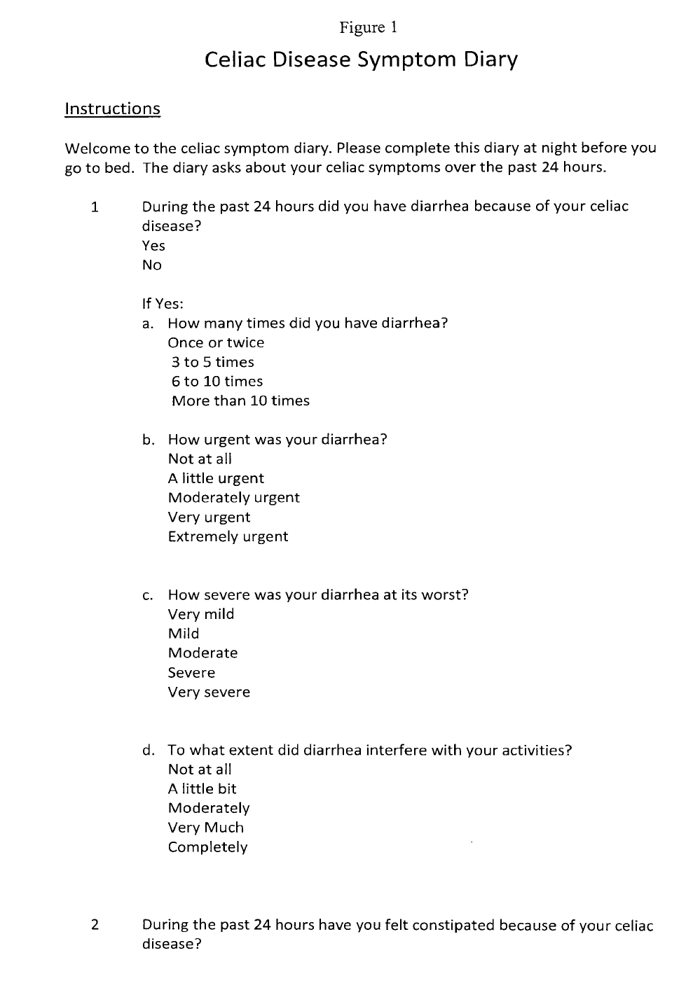

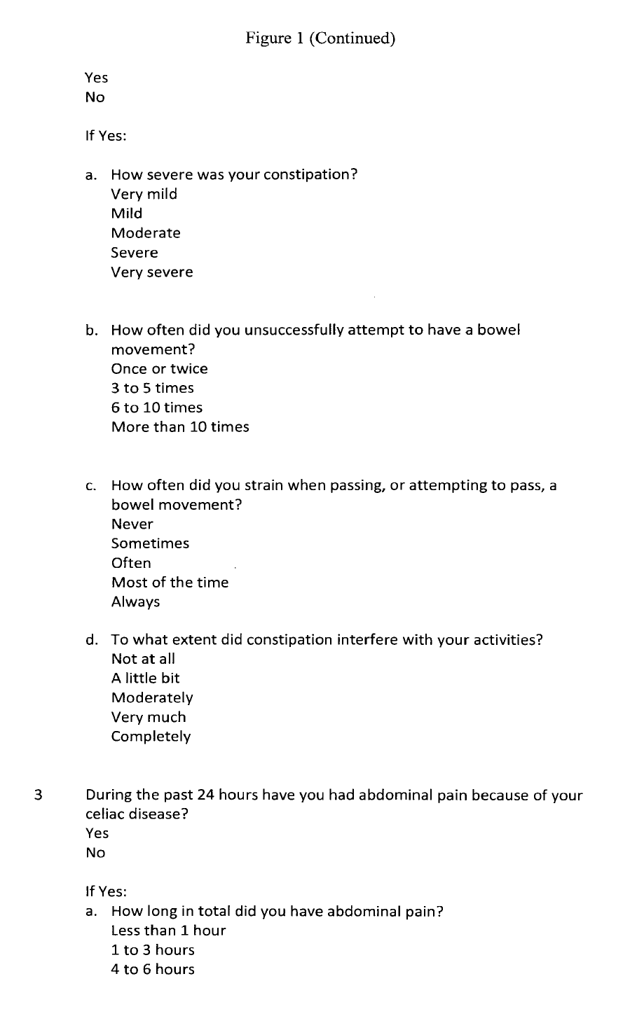

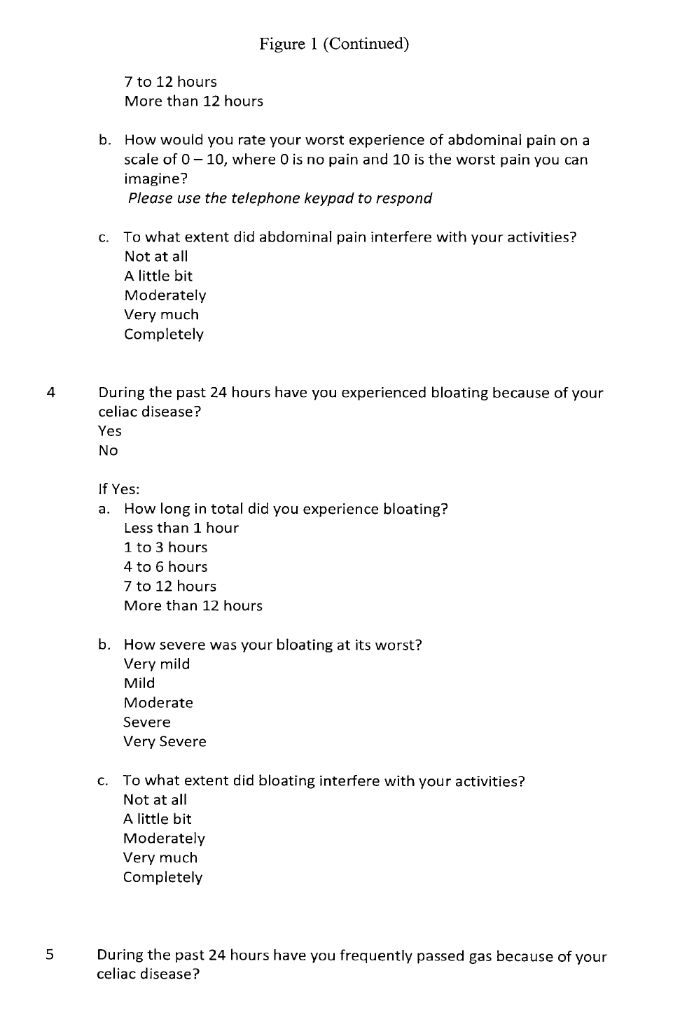

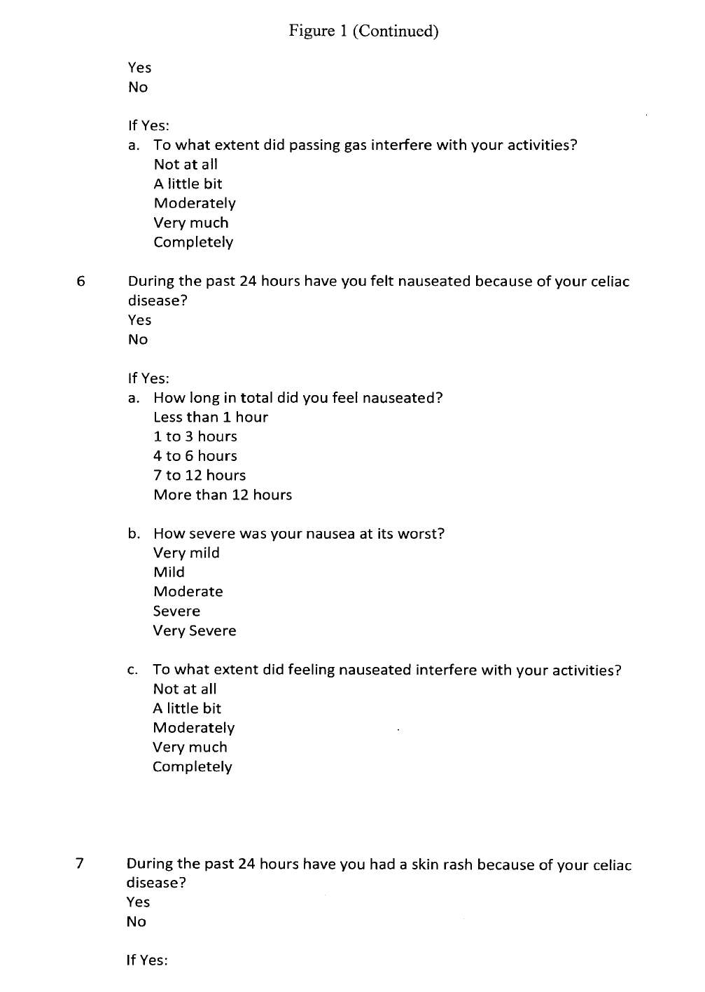

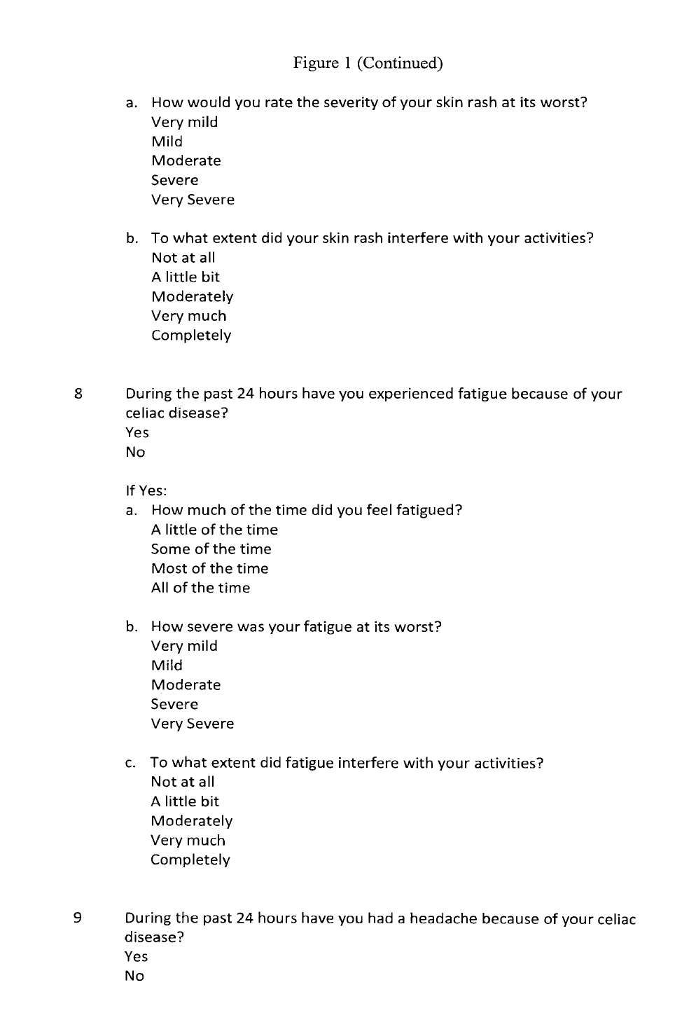

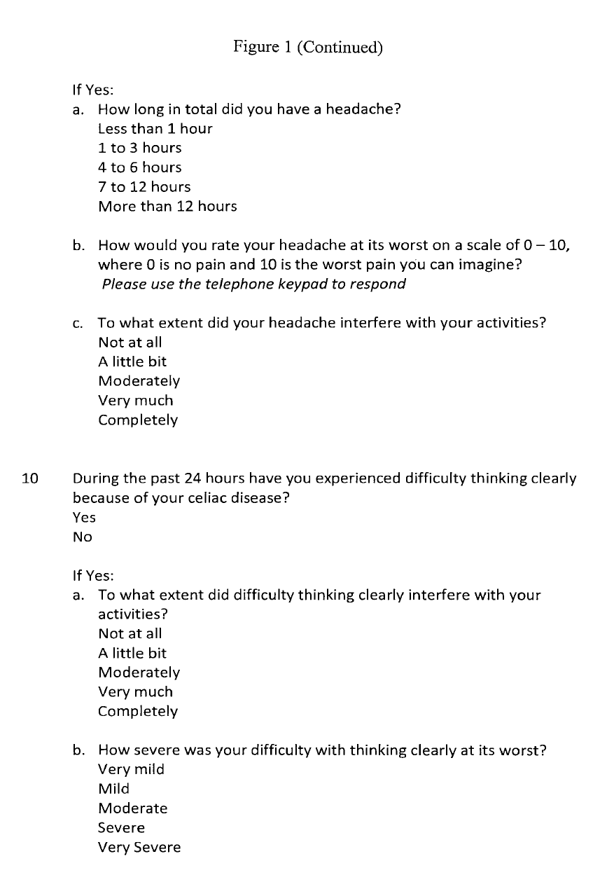

3. The PRO of claim 1 essentially as shown in FIG. 1.

4. A patient reported outcome questionnaire (PRO) for patients who have or are suspected of having celiac disease, gluten intolerance, or gluten sensitivity in the form of weekly questionnaire that prompts the subject to provide a rating of the extent to which the subject's symptoms have affected the subject's daily life, social activities, emotional wellbeing, and physical functioning.

5. The PRO of claim 4 that further prompts the subject to provide the rating in the form of one of five possible rates, from “not at all” to “completely”, with intermediate rates of “a little”, “moderately”, and “very much”.

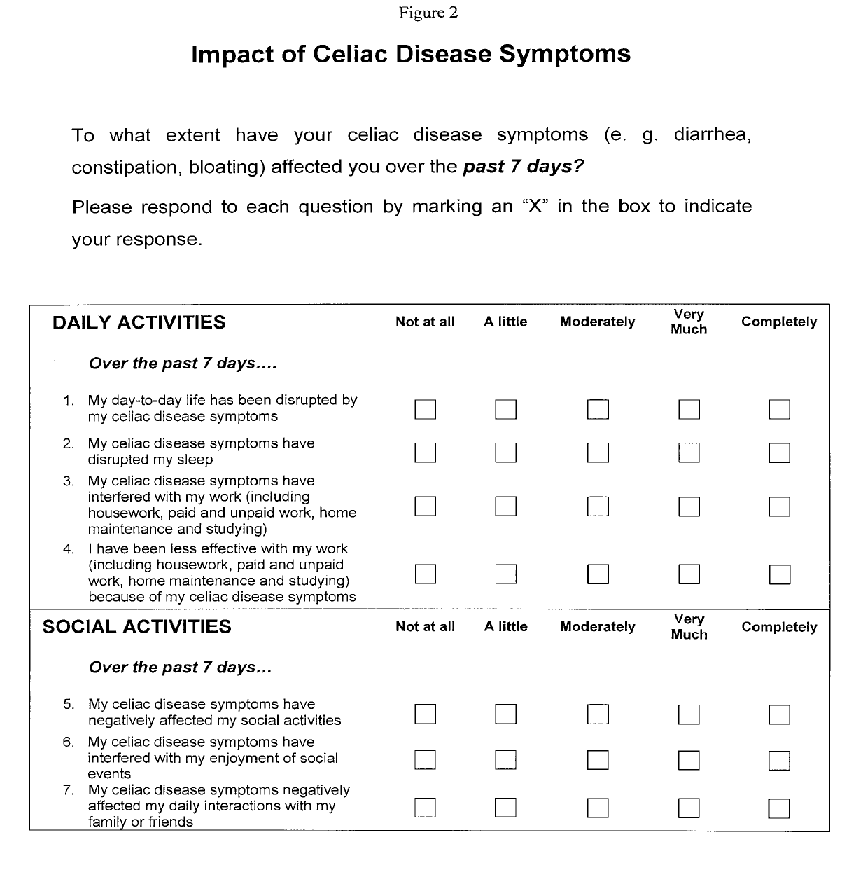

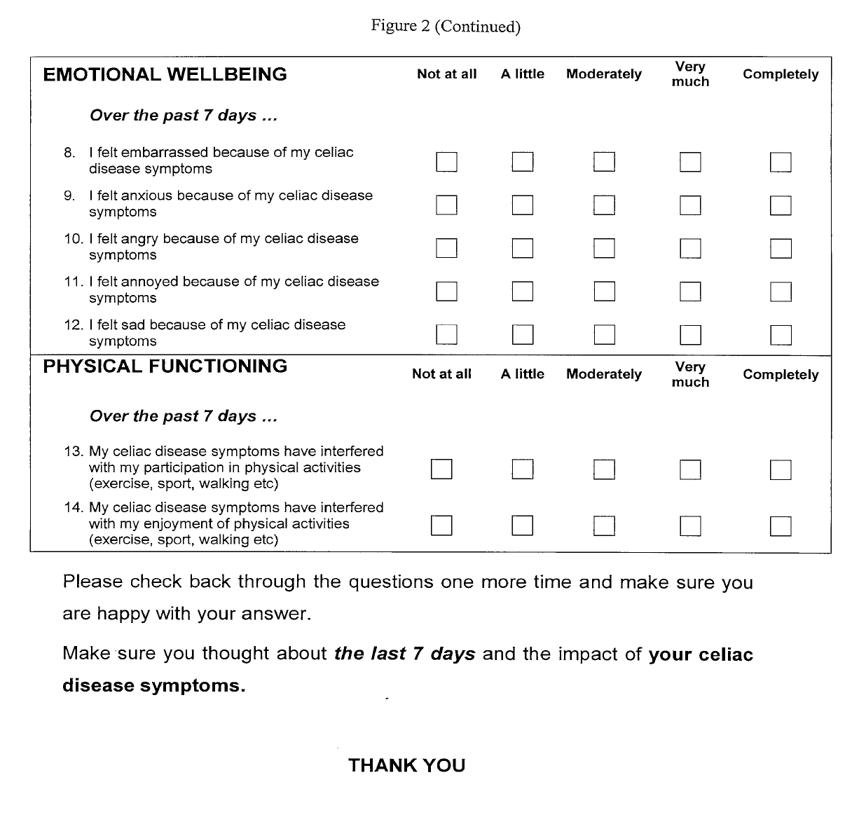

6. The PRO of claim 5 essentially as shown in FIG. 2.

7. A patient reported outcome questionnaire (PRO) for patients on a gluten free diet in the form of weekly questionnaire that prompts the subject to provide a rating of the extent to which the subject's adherence to a GFD has affected the subject's quality of life in areas such as dietary choices and access to foods of interest to the subject, ability to attend social events, emotional wellbeing.

8. The PRO of claim 7 that further prompts the subject to provide the rating in the form of one of five possible rates, from “not at all” to “completely”, with intermediate rates of “a little”, “moderately”, and “very much”.

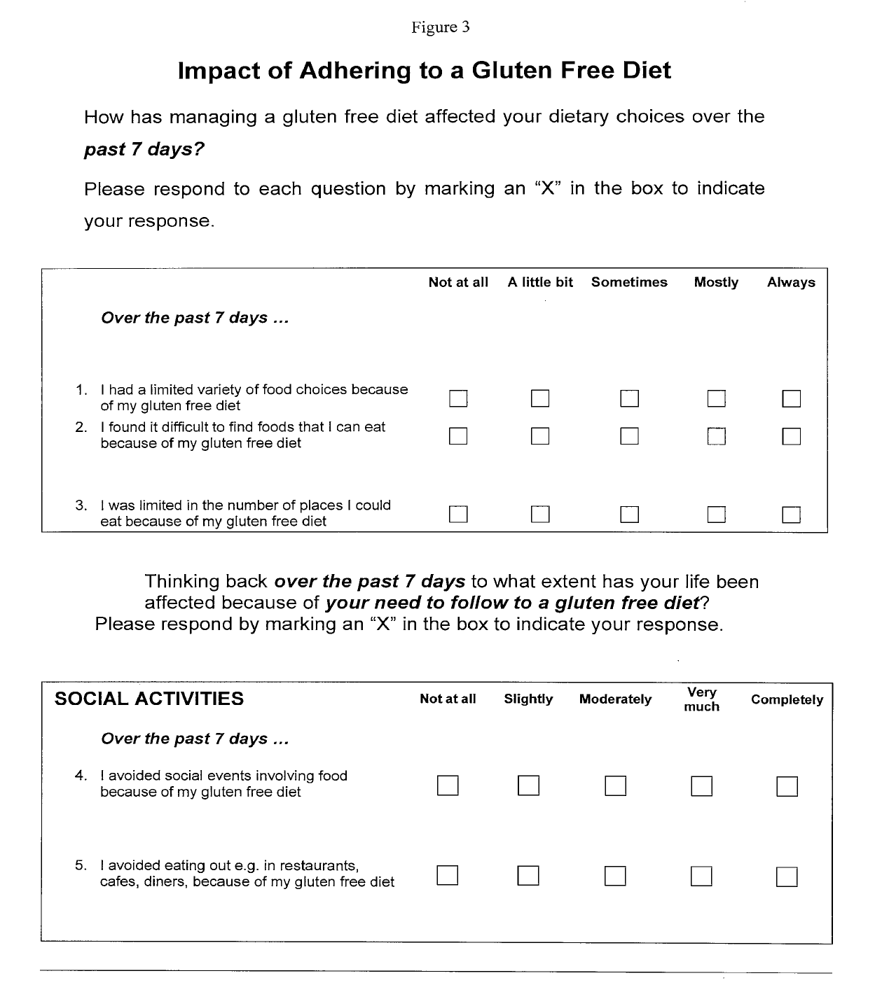

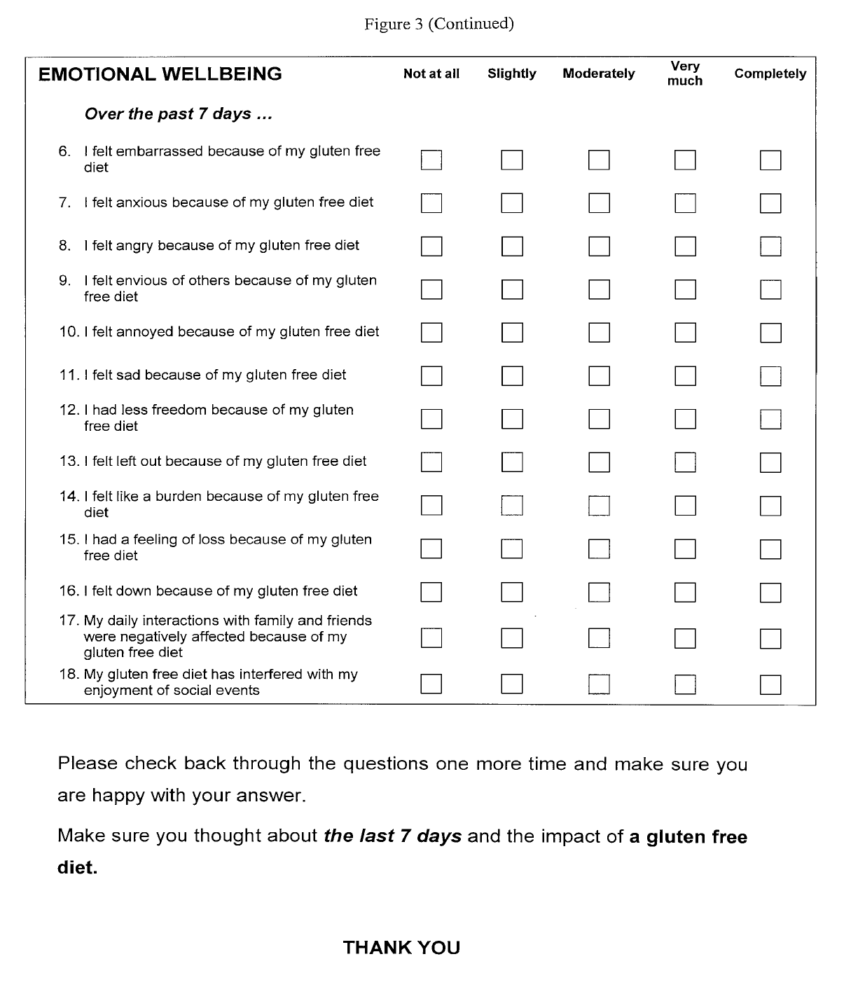

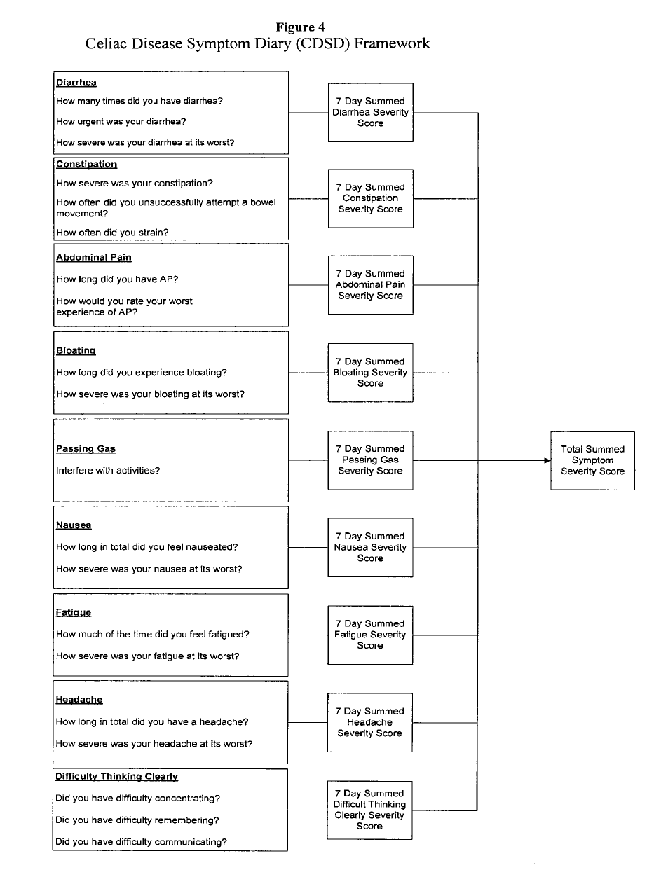

9. The PRO of claim 8 essentially as shown in FIG. 3.

10. A method for assessing celiac disease symptoms in a celiac disease patient, said method comprising the steps of providing the patient with a PRO selected from the group consisting of the PROs of claim 1 or 4; instructing the patient to complete the PRO; collecting the questionnaires; and generating an assessment of said symptoms from said questionnaires.

11. The method of claim 10, wherein the celiac disease patient is a symptomatic celiac disease patient on a gluten-free diet (GFD).

12. The method of claim 11, wherein the patient has gastrointestinal (GI) symptoms.

13. The method of claim 12, wherein the patient additionally has non-GI symptoms.

14. The method of claim 11, wherein the patient's symptoms and selected from the group consisting of abdominal pain, bloating, constipation, diarrhea, fatigue, flatulence, headache, nausea, skin rash, and problems thinking clearly.

15. The method of claim 11, wherein the patient is instructed to complete the PRO once a day.

16. The method of claim 15, wherein the patient is instructed to complete the PRO for at least two weeks.

17. The method of claim 15 wherein the patient is instructed to complete the PRO for at least four weeks.

18. The method of claim 15 wherein the patient is instructed to complete the PRO for at least six weeks.

19. The method of claim 10, further comprising the step of administering to said patient a glutenase capable of degrading gluten.

20. The method of claim 19, wherein said glutenase comprises a prolyl endopeptidase (PEP).

21. The method of claim 20, wherein the glutenase comprises ALV001 (a modified recombinant version of the proenzyme form of cysteine endoprotease EP-B2 from barley) and ALV002 (a modified recombinant version of a prolyl endopeptidase from the bacterium Sphingomonas capsulata (SC-PEP).

22. The method of claim 20 wherein the glutenase is ALV003, an orally administered, fixed dose, 1:1 ratio by weight combination of ALV001 and ALV002.
